(19) United States
(12) Patent Application Publication
(10) Pub. No.: US 2022/0170652 A1
(43) Pub. Date: Jun. 2, 2022

(54) SYSTEM AND METHOD FOR DISINFECTION OF AIR

(71) Applicant: Aditya Mathur, Porter Ranch, CA (US)

(72) Inventor: Aditya Mathur, Porter Ranch, CA (US)

(21) Appl. No.: 17/539,869

(22) Filed: Dec. 1, 2021

Related U.S. Application Data
(60) Provisional application No. 63/119,651, filed on Dec. 1, 2020.

(51) Int. Cl. F24F 8/22; F24F 7/007; F24F 11/74

(52) U.S. Cl. CPC F24F 8/22; F24F 11/74; F24F 7/007

(57) ABSTRACT

A disinfection system including an air mover, wherein the air mover is configured to direct air flow; an inlet filter implement; an inlet flow control valve; a decontamination chamber apparatus, in which the decontamination chamber apparatus comprises an inlet section or port, an inner wall, and an outlet section or port; a UV light source, wherein the UV light source is configured to produce light in the UVC wavelength, wherein the UV light is configured to disinfect air that passes through the decontamination chamber apparatus; an outlet flow control valve, wherein the outlet flow control valve is configured to recirculate the disinfected air; and wherein the decontamination chamber apparatus is configured to be operable for impeding or obstructing the flow of air.

### CROSS-REFERENCE TO RELATED APPLICATIONS

The present Utility patent application claims priority benefit of the U.S. provisional application for patent Ser. No. 63/119,651 “Controlled Air Flow Disinfection System”, filed on 1 Dec. 2020, under 35 U.S.C. 119(e). The contents of this related provisional application are incorporated herein by reference for all purposes to the extent that such subject matter is not inconsistent herewith or limiting hereof.

### INCORPORATION BY REFERENCE OF SEQUENCE LISTING PROVIDED AS A TEXT FILE

Not applicable.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to disinfection systems. More particularly, certain embodiments of the invention relate to UVC disinfection system of air/water.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

Coronavirus disease 2019 (COVID-19) was first reported in December 2019 and then quickly characterized as a pandemic by World Health Organization. Transmission of beta coronavirus which causes COVID-19, is believed to be both through direct contact and airborne routes. Given the rapid spread of the disease, it may be of clear importance to explore practical mitigation technologies that may deactivate the airborne viruses. The severity of the 2020 COVID-19 pandemic warrants the rapid development and deployment of effective countermeasures to reduce person-to-person transmission in infectious environments. Direct contact transmission may be managed by personal hygiene and distancing. Air borne transmission may pose a threat to “susceptible population” as well to “essential workers who spend many hours in virus rich air.”

The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that mechanical methods such as filtration masks and/or face shields are not totally effective deterrent against COVID-19. Insufficiency of filtration masks and/or face shields for COVID-19 has been exhibited through the spread of infections in medical workers at several medical facilities around the world. Ultraviolet (UV) light may be a direct antimicrobial and its effectiveness against different strains of viruses is well established. Several UV disinfectant systems are in use for sterilization of medical instruments and appliances at many medical facilities. Several portable air cleaning systems and some heating and cooling ducting systems installed in residential and commercial buildings may include UV light sources, which are used to “disinfect” walls or other internal surfaces of ducting where moisture may condense. UV radiation has effectively been used for decades to reduce the spread of bacteria, such as tuberculosis. For this reason, UV lamps are often called “germicidal” lamps. UVC radiation has been shown to destroy the outer protein coating of the SARS-Coronavirus, which is a different virus from the current SARS-CoV-2 virus. The destruction of the outer protein coating leads to inactivation of the virus. UVC radiation may also be effective in inactivating the SARS-CoV-2 virus, which is the virus that causes the Coronavirus Disease 2019 (COVID-19). Currently, there is limited published data about the wavelength, dose, and duration of UVC radiation required to inactivate the SARS-CoV-2 virus. In these systems, no attempt is made to effectively disinfect air moving through air handling systems.

In nature, solar UV radiation may act as the principal source for deactivation of viruses. Sunlight may produce three main types of UV rays including UVA, UVB, and UVC. UVA rays have the longest wavelengths, followed by UVB, and UVC rays which have the shortest wavelengths. While UVA and UVB rays may be transmitted through the atmosphere, most UVC and some UVB rays may be absorbed by the Earth's ozone layer. Most UV rays present in the environment are UVA with a small amount of UVB. UVC radiation from the sun may not reach the earth's surface because it is blocked by the ozone layer in the earth's atmosphere. The UVC wavelengths that are most effective (about 300 nm and shorter) may not be present in solar radiation reaching the earth's surface. The ability to deactivate viruses may be reduced at longer wavelengths. Due to lack of UVC radiation, several viruses may, and do, persist for long duration of time in the natural environment. Longer wavelengths of UVA and UVB may allow some group of viruses to remain active and spread more rapidly. Corona virus may be one of these viruses. One of the ways that viruses may be exposed to UVC radiation is from typical artificial sources such as UV LED lamps.

Conventional lamps may emit very specific UVC wavelengths (like 254 nm or 222 nm), or they may emit a broad range of UV wavelengths. Some lamps may also emit visible and infrared radiation. The wavelengths emitted by the lamp may affect the lamp's effectiveness at inactivating a virus and may impact the health and safety risks associated with the lamp. Some lamps may emit multiple types of wavelengths. Typically, the most common type of lamp used to produce UVC radiation was the low-pressure mercury lamp, which has its main (>90%) emission at 254 nm. Other wavelengths may also be produced by this type of lamp. There are other lamps available that emit a broad range of UV wavelengths, but also emit visible and infrared radiation. Light-emitting diodes (LEDs) that produce UV radiation are also becoming more commonly available. Typically, LEDs emit a very narrow wavelength band of radiation. Conventional UV LEDs may have peak wavelengths at approximately 265 nm, 273 nm, and 280 nm. One advantage of LEDs over low-pressure mercury lamps is that they contain no mercury. The small surface area and higher directionality of LEDs may make them less effective for germicidal applications.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is an illustration of an exemplary disinfection system, in accordance with an embodiment of the present invention;

FIG. 2a is an illustration of exemplary components of a disinfection system, in accordance with an embodiment of the present invention;

FIG. 2b is a front view illustration of an exemplary decontamination chamber apparatus of a disinfection system, in accordance with an embodiment of the present invention; and

FIG. 3 is an illustration of an exemplary graph showing an impact of UVC light on virus, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settled law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [“substantially”] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit —“about 10%” —the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” include the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred, or induced by any pattern(s) of description, embodiments, examples, or referenced prior-art that may (or may not) be provided in the present patent.

References to “end user”, or any similar term, as used herein, is generally intended to mean late-stage user(s) as opposed to early-stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of. or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising” And “contain” and variations of them—Such terms are open-ended and mean “including but not limited to”. When employed in the appended claims, this term does not foreclose additional structure or steps. Consider a claim that recites: “A memory controller comprising a system cache . . . .” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn.112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

All terms of exemplary language (e.g., including, without limitation, “such as”, “like”, “for example”, “for instance”, “similar to”, etc.) are not exclusive of any other, potentially, unrelated, types of examples; thus, implicitly mean “by way of example, and not limitation . . . ”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of,” where one of these three terms is used herein, the disclosed and claimed subject matter may include the use of either of the other two terms. Thus, in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Moreover, any claim limitation phrased in functional limitation terms covered by 35 USC § 112(6) (post AIA 112(f)) which has a preamble invoking the closed terms “consisting of,” or “consisting essentially of,” should be understood to mean that the corresponding structure(s) disclosed herein define the exact metes and bounds of what the so claimed invention embodiment(s) consists of, or consisting essentially of, to the exclusion of any other elements which do not materially affect the intended purpose of the so claimed embodiment(s).

Devices or system modules that are in at least general communication with each other need not be in continuous communication with each other, unless expressly specified otherwise. In addition, devices or system modules that are in at least general communication with each other may communicate directly or indirectly through one or more intermediaries. Moreover, it is understood that any system components described or named in any embodiment or claimed herein may be grouped or sub-grouped (and accordingly implicitly renamed) in any combination or sub-combination as those skilled in the art can imagine as suitable for the particular application, and still be within the scope and spirit of the claimed embodiments of the present invention. For an example of what this means, if the invention was a controller of a motor and a valve and the embodiments and claims articulated those components as being separately grouped and connected, applying the foregoing would mean that such an invention and claims would also implicitly cover the valve being grouped inside the motor and the controller being a remote controller with no direct physical connection to the motor or internalized valve, as such the claimed invention is contemplated to cover all ways of grouping and/or adding of intermediate components or systems that still substantially achieve the intended result of the invention.

A description of an embodiment with several components in communication with each other does not imply that all such components are required. On the contrary a variety of optional components is described to illustrate the wide variety of possible embodiments of the present invention.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In the following description and claims, the terms “coupled” and “connected,” along with their derivatives, may be used. It should be understood that these terms are not intended as synonyms for each other. Rather, in particular embodiments, “connected” may be used to indicate that two or more elements are in direct physical or electrical contact with each other. “Coupled” may mean that two or more elements are in direct physical or electrical contact. However, “coupled” may also mean that two or more elements are not in direct contact with each other, but yet still cooperate or interact with each other.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

In some embodiments of the present invention and variations thereof, relate to disinfection systems and methods. In one embodiment of the present invention, disinfection system may disinfect air. Disinfecting air may require a delivery of a critical amount of UVC energy to the air mass. In some embodiment, a UVC disinfectant system may be used for sterilization of medical instruments and appliances at medical facilities. Disinfection may be achieved by putting objects to be disinfected on a platform in UV light for a length of time. UVC disinfection of solid objects is generally thought of as one which requires a “line of sight” exposure. Solid objects to be disinfected may need to be turned over so that all sides of the objects are disinfected. This may be because photons do not reach in shadows cast by solid object to be disinfected. In another embodiment, UVC based liquid disinfection systems may disinfect liquids such as water disinfection.

Components of the air disinfecting system may comprise, but not limited to, a UVC light source and means to create a near uniform distribution of light in a disinfecting region. The air to be treated slowly moves in the disinfecting region from the inlet section/port to an outlet section/port and/or it may be held for a length of time at room pressure or at higher pressure in the disinfecting region and then released. Any UVC light source may be used to create the photons. For portable air disinfection systems, UVC LED devices are devices that require low voltage power supplies and offer high efficiency of UVC generation. Significant improvements in LED technology are continuing and new generations of LEDs may be developed and easily incorporated in the system.

In another embodiment, a UVC exposure area of a decontamination chamber may surround the air mass with, but not limited to, a layer of UVC reflecting coating or material for reflecting UVC light. A thin layer of a conducting material such as, but not limited to, Aluminum may be deposited on the wall of the chamber, a conducting sheet such as Aluminum foil may be glued to the chamber walls, a thin layer of Aluminum may be plated on the walls, or a special synthetic material such as sintered PTFE (Polytetrafluoroethylene) may be used for this purpose. The reflecting layer may be covered with another dielectric material to protect the reflection layer from environmental degradation or the reflection of UVC may be achieved by using a stack of dielectric layers with different dielectric constants designed to act as a reflector. For example, alternating layers of magnesium fluoride and fluoropolymers.

In an embodiment, the purpose of the reflective layer is to reflect a large portion of incident UVC photons. But all the photons do not need to be sent directly back towards the source. Instead, different micro-patches of the reflective layer may reflect and distribute photons in different directions. This objective may be achieved by creating a mechanically rough surface (like the surface of an orange) but coated with highly UVC reflective mirror film. When a UVC photon arrives at the surface, it may be reflected for disinfection. The direction of the reflection may be determined by microscopic features (micro-patches) of the local surface. Alternatively, this objective may also be achieved by using surfaces which vary in the grain structure at microscopic level. The “reflecting surfaces” may be needed to enhance diffusive reflectivity. The level of roughness and variation such as a texture, pyramids and grooves, or roughness to scatter light. This can be further modified by using other patterns or shapes to optimally scatter and reflect the light. in the local finish or features may be used to control the actual distribution pattern of the reflected photons. In alternative embodiment, a layer of sintered PTFE (Polytetrafluoroethylene) or other materials which offer a similar ability to reflect the photons and create a uniform environment of photons are equally suitable for this application. The elements of the decontamination chamber wall design may allow creating a shadow less brightly illuminated space which may be a crucial part of disinfection of air.

In additional embodiment, the flow of air and the nature of the air flow in the disinfection area may play a crucial role in enhancing the chances of interaction between air borne viruses and UVC photons. The objective is to have a steady but near zero velocity or slow movement of air from the inlet section/port to the outlet section/port of the decontamination chamber. A low average velocity may be provided in the direction of the outlet port. Mechanical means may be used to create reflection of air molecules in multiple directions to increase the actual travel path length for air (and virus molecules). In one version of the decontamination chamber design, an air velocity control and UVC exposure may be combined in a single physical area or volume. Combining the air velocity control and UVC exposure may be achieved by tightly packing an “active area” in the decontamination chamber with UVC transparent materials such as, but not limited to, balls, fiber, and/or stack of perforated thin wafer glass/plastic baffles suitably spaced and rotated with respect to each other to create a stagger in air passage through the perforations. The UVC transparent material may be made of, but not limited to, a variety of UVC transparent glasses such as Quartz or Fused Silica or PTFE (Polytetrafluoroethylene) or another suitable material. The “active disinfection area” may maintain a steady air flow by creating a pressure gradient across a decontamination chamber region. Filters may be disposed along a length of the decontamination chamber which effectively create various sections within and along the length of the decontamination chamber to further control the flow of air being disinfected.

Disinfection of air (being compressible) requires development of flow control techniques to provide sustained exposure of air being disinfected to stationary UVC sources. The tools used to achieve flow control and hence control the time for UVC exposure and air interaction may include, but not limited to, management of air pressure and flow via defined impedances (e.g., filters), distributed impedances (e.g., powders and fiber packing of interaction space), air moving devices and flow control valves. In some embodiment, the air mass and UV-C interaction time may be controlled by holding the air mass in the UV-C illuminated area by using programmable on/off valves at the inlet and outlet port/section of the decontamination chamber. The air inlet and outlet valves as well as the UV-C light source are controlled by, but not limited to, a computer, a program and processor, an embedded controller, etc. The flow of air may be further slowed by obstructing the path of air flow in the UV-C illuminated areas. The obstruction is achieved by using any combination of the following materials such as but not limited to UV-C transparent wool, foam, particles, balls, beads, or perforated disks.

In additional embodiment, the air disinfection system may include multiple air decontamination chambers. The multiple air decontamination chambers may be connected in series or parallel configured to enhance the level of disinfection. Alternately, the multiple air decontamination chambers may be connected in combination of series and parallel connections configured to enhance the level of disinfection.

The present invention will now be described in detail with reference to embodiments thereof as illustrated in the accompanying drawings.

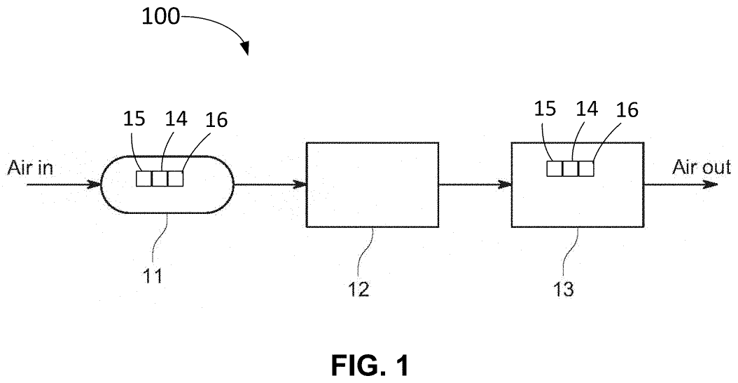

FIG. 1 is an illustration of an exemplary disinfection system 100, in accordance with an embodiment of the present invention. In one embodiment, disinfection system 100 may include but not limited to an air collection and air moving device 11, a decontamination chamber implement 12, and a delivery subsystem 13. Air collection and air moving device 11 may comprise, but not limited to, a fan and/or air compressor, flow control valves, filters, controller, etc. Air collection and air moving device 11 may be programmable to automatically turn the fan and/or air compressor on/off, start the disinfection process, and/or control air flow with controller 14. Controller 14 may be coupled to a timer device (15). Timer device (15) may be separate from or integrated with controller 14 for determining UV-C interaction time with the air mass in the UVC illuminated area. Controller 14 may include but not limited to computers, embedded controllers, programmable flow controllers that can be programmed by user input 16, etc. User input 16 may comprise keypad, keyboard, etc. Decontamination chamber apparatus 12 may include one or more decontamination chambers that houses a UVC light source where disinfection of air happens in the chamber. UVC light source may include but not limited to, at least one of, UVC LEDs, Hg arc lamps, pulsed Xenon lamps, K-Br lamps. Delivery subsystem 13 may include, but not limited to ducts, pipes for larger scale application, and tubes transports and distributes the disinfected air to a point of usage. This can be further modified by utilizing other means of air transport. In some embodiment, multiple decontamination chamber apparatus 12 may be connected in series and/or parallel to enhance the level of disinfection before distributing the disinfected air to the point of usage.

In another embodiment, disinfection system 100 may include mechanisms to drench volumes of air in UVC light. The concept of “line of sight” commonly used in the context of UVC disinfection, and mentioned above, may not extend well to air disinfection because air is transparent to light and air does not cast shadows. What is needed are means to create and maintain a high level of UVC flux. Creation of a UVC rich environment and maintaining a high level of UVC flux, in the decontamination chamber, is another aspect of the disclosure. To maintain a high level of UVC flux the walls of the chambers are designed reflect the UVC light and the light sources are chosen to provide the necessary number of photons.

Disinfection of air (being compressible) may require development of flow control techniques to provide sustained exposure of air being disinfected to UVC. In one embodiment, the tools used to achieve air flow control and control of the exposure time of air to UVC and air interaction may include, but not limited to, management of air pressure and air flow via defined impedances including but not limited to filters, distributed impedances including but not limited to powders and fiber packing of interaction space, air moving devices including, but not a limitation, fans and/or air compressors, and flow control valves to reduce the rate of air flow.

Impact of UVC light on virus: UV radiation may disable viruses by chemically modifying the genetic material of viruses. UVC (about 200-300 nm UV radiation) photons break a critical bond in the structure of the nucleic acid that removes the duplicating ability of virus molecules. DNA-containing viruses are more sensitive to this damage than RNA-containing viruses, but both types of viruses may be disabled. The key parameters for this process are: photon energy, the number of photons present, and the number of critical bonds present in the virus. Presence of higher number of photons in the environment of the virus and length of exposure of the virus to the photons, increase the level of disinfection.

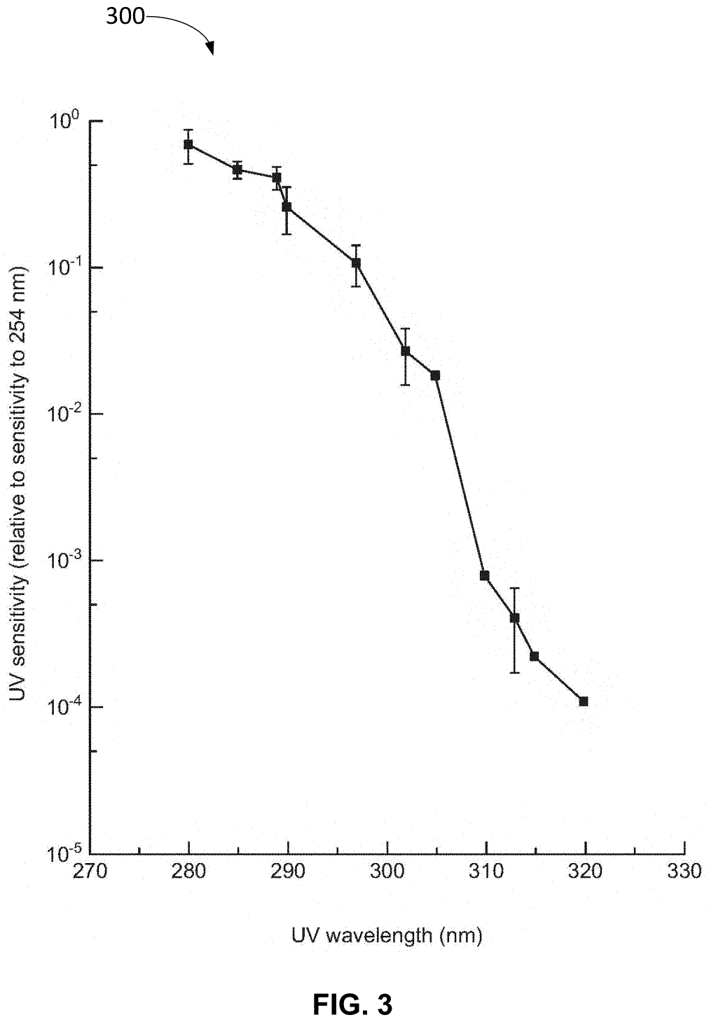

In nature, solar UV radiation may act as a principal source for deactivation of viruses. Referring to FIG. 1 and FIG. 3, the wavelengths that may be most effective (about 300 nm and shorter) are not present in solar radiation reaching the earth's surface. The ability to deactivate viruses may be reduced at longer UV wavelengths as shown in FIG. 3. At longer UV wavelengths, several viruses may, and do, persist for a longer duration of time in a natural environment. The longer UV wavelengths may allow a group of viruses to remain active and spread more rapidly. Corona virus is one type of these viruses. Since, UVC wavelengths may not be present in sunlight (solar UV radiation) that reaches the earth's surface, man-made UVC sources may be required for disinfection systems.

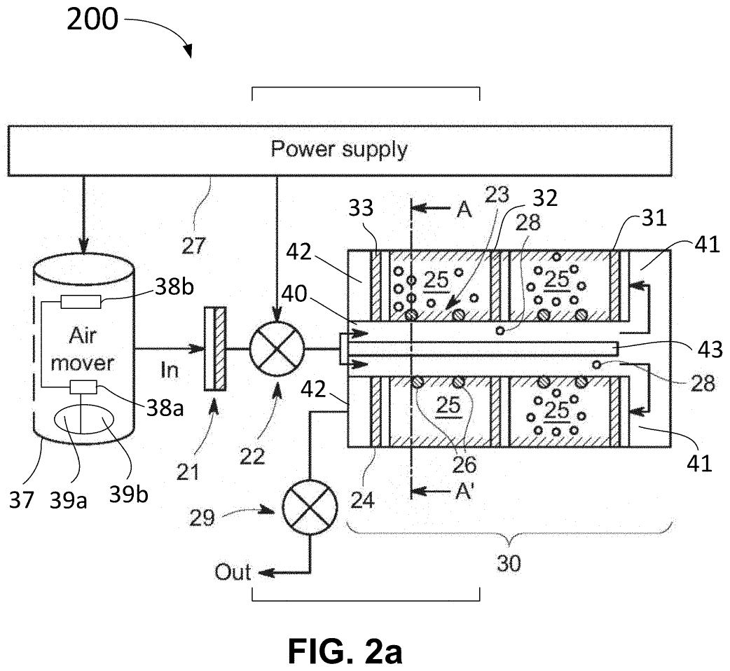

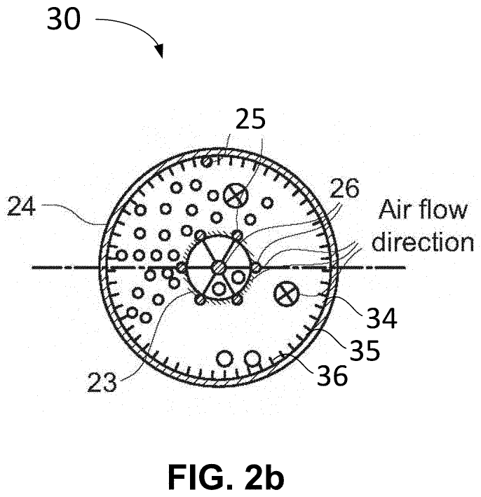

FIG. 2a is an illustration of exemplary components of a disinfection system, in accordance with an embodiment of the present invention. In one embodiment of the present invention, disinfection system 200 may include, but not limited to, an air mover (37), an inlet filter instrument such as HEPA filters or modify with other types of common air filters (21), an inlet flow control valve such as mass flow controller or a needle valve (22) a decontamination chamber apparatus (30), and an outlet flow control valve (29). Air mover (37) may include fan (38a) for directing air into decontamination chamber apparatus (30) and/or air compressor (38b) for forcing air into decontamination chamber apparatus (30) and build pressure. Air mover (37) may further include a programmable controller (39a) to turn the fan and/or air compressor on/off, start the disinfection process, and/or control air flow at predetermined times and/or conditions. Timer device (39b) may be separate from or integrated with controller (39a) for determining UVC interaction time with the air mass in the UVC illuminated area. Controller (39a) may include but not limited to computers, processors, embedded controllers, programmable flow controllers, etc. Controller (39) may turn on/off inlet flow control valve (22) and outlet flow control valve such as a mass flow controller or needle valve or this can be modified by using other types of flow control valves (29) to control the air flow going in and out of decontamination chamber apparatus (30) Decontamination chamber apparatus (30) may include one or more decontamination chambers, where each decontamination chamber may comprise one or more sections of active decontamination area (25) separated with filters along a length of each decontamination chamber. One or more inner tube segments (28) are configured to feed air into each decontamination chamber. Divider part (43) separates the decontamination chambers and the inner tube segments.

Filter implement (24) may be disposed at different sections along a length of each decontamination chamber, creating active decontamination areas (25) and to produce a predetermined amount of pressure drop on each active decontamination area (25). Filter implement (24) may include one or more filters disposed at an entrance portion (31), a middle portion (32), and an end portion (33) of each decontamination chamber. Each decontamination area (25) may be filled with but not limited to UVC transparent glass balls (23) or other materials such as powdered Quartz, Quartz wool, UV-C transparent wool, foam, particles, balls, beads, or perforated disks configured to impede, obstruct, or hinder the flow of air. The inner walls of each decontamination chamber or active decontamination area (25) may include layers of highly reflective materials. An array of LEDs (26) are mounted on a portion of the inner wall. The reflective layers may comprise reflection coatings and patterns that are designed to create a diffused UVC photon environment for disinfecting air inside the decontamination areas (25). Another feature of the inner walls may be that these are finished with special surface features such as a texture, pyramids and grooves, or roughness to scatter light to create further resistance to air flow near the boundary of the walls. The space outside the decontamination area (25) may include electrical wiring to supply power to the LEDs as well as cooling fins, the metal protrusions that dissipate heat as commonly found in CPU and electronic cooling components to remove heat produced by the array of LEDs. The whole unit may be powered by power supply (27). Power supply (27) may include but not limited to AC power supply, DC power supply, rechargeable batteries, etc. The effectiveness of the disinfection process may be controlled by the total number of photons (light produced) by LED array similar to the dimming switches in use with conventional or household lighting (26), the ability of the reflection coatings and patterns on the inner wall of decontamination areas (25) to diffuse UVC, the time spent by air in the decontamination areas (25) which are filled with glass balls/powder (23), and filter implement (24).

In practice, air enters through entry port (40) and may travel to the end of inner tube segment (28). The air may enter inlet section/port (41) of the decontamination chamber. The air passes through each active decontamination area (25) and may be slowed or stopped for a predetermined amount of time at each active decontamination area (25) for UVC exposure. Until the disinfected air reaches outlet section/port (42). The disinfected air leaves outlet section/port (42) of decontamination chamber apparatus (30) via outlet flow control valve (29) for recirculation or sent to the user via the air delivery system (13, FIG. 1).

FIG. 2b is a front view illustration of an exemplary decontamination chamber apparatus (30) of a disinfection system, in accordance with an embodiment of the present invention. In one embodiment of the present invention, decontamination chamber apparatus (30) may include UVC transparent glass balls (23) or other materials such as powdered Quartz or Quartz wool, to impede the flow of air. Filter implement (24) may include one or more filters disposed at different sections along the length of the decontamination chamber apparatus (30). The dispersion of the filters along entrance portion (31), middle portion (32), and end portion (33) are designed to produce certain amount of pressure drop between the different decontamination areas (25). Inner walls (34) may comprise reflective layers (35) that are designed to create a diffused UVC photon environment from incident UVC light inside the decontamination areas (25). Reflective layers (35) may include but not limited to layers of conducting materials such as, but not limited to, Aluminum, Aluminum foil, and/or a special synthetic material such as sintered PTFE (Polytetrafluoroethylene). Thin layers of Aluminum may be deposited or plated on a surface of inner walls (34) of decontamination area (25). Or, Aluminum foil may be glued to inner walls (34). In an alternative embodiment, the special synthetic material sintered PTFE (Polytetrafluoroethylene) may be glued or plated to inner walls (34). Reflective layer (35) may be covered with dielectric material (36) to protect reflective layer (35) from environmental degradation. In other alternative embodiment, a stack of dielectric layers (36) with different dielectric constants may be glued or plated to inner walls (34) to act as a reflector.

The average pressure in the decontamination areas (25) or the decontamination chamber (30) may be raised so that it is higher than atmospheric pressure with the air compressor and by adding “air reservoirs”, such as compressed or expandable air tanks, and flow control valves at the input and output ports of the system. This may allow an increase of the amount of room air that may be disinfected, which may follow directly from ideal gas pressure/volume and temperature relationships—the volume throughput scales directly with pressure.

FIG. 3 is an illustration of an exemplary graph showing an impact of UVC light on virus, in accordance with an embodiment of the present invention. Referring to FIG. 3, UV radiation may disable viruses by chemically modifying the genetic material of viruses. UVC (at about 200-300 nm UV radiation) photons may break a critical bond in the structure of the nucleic acid that removes the duplicating ability of virus molecule. DNA-containing viruses are more sensitive to this damage than RNA-containing viruses, but both types of viruses may be disabled. The key parameters for this process are: photon energy, the number of photons present and the number of critical bonds present in the virus. The presence of higher number of photons in the environment of the virus and the length of exposure of the virus to the photons, may increase the level of disinfection.

In one embodiment, Hg arc lamps may be used as source of UVC light. An electrical arc may be created in low vapor pressure of mercury and the arc may emit light at about 253.7 nm. In another embodiment, pulsed Xenon lamps and K-Br lamps with peak emission at about 220-230 nm may also be used as a source of UVC light. The effect of approximately 222 nm radiation on viruses using a K-Br lamp may be as effective as Hg arc lamp radiation for COVID-19 virus. About two hundred eighty (280) nm photons may have sufficient energy to break the critical bonds which are involved in the “duplicating ability” of the virus.

In some embodiment, LEDs may be used as source of UVC light. UVC LED sources may offer long life, high efficiency of conversion and produce a high intensity illumination and may use low voltage for operation. UVC LEDs which produce about ˜270 nm UVC may be used as source of UVC light. As shown in FIG. 3, the effectiveness of approximately 270 nm of UV LED light is about 90% of a Hg 254 nm light. Ease of handling of LEDs and safety of production lines are some of the main reasons to choose LEDs as the source of UVC generation. But other UVC lamps such as Hg lamps and K-Br lamps or any other source of UVC may also be used. Wide scale adoption of UVC LEDs for disinfection of air may drive the development of more efficient and shorter wavelength LEDs.

The next variable in determining the effectiveness of a disinfection system is the total flux of light which may be available and the length of time microorganisms and/or viruses are exposed to the UVC light. In one embodiment, total flux of light and the length of time may control the total dosage which is delivered to the microorganisms/virus population. As a measure of “flux” or “strength of light field”, one may use total number of UV photons per unit volume. It may be difficult to measure this quantity. In other embodiment, the amount of photon which are delivered by a light field to a calibrated sensor is used as a probe. The number represents total photon energy incident on the sensor in terms of Watts/cm2. The actual light flux or photon dosage present may be directly proportional to the measured number. Length of exposure time may be directly estimated as the average time taken by air to move through the decontamination chamber. Mass flow meters and total volume of air in the decontamination chamber may be used to calculate the exposure time.

The nucleic acid within a virus particle may play a crucial role in virus inactivation. The number of bases in the DNA or RNA of the virus may be important for determining its sensitivity to UV inactivation, because the higher the number of target bases, the more likely the genome will be damaged at a given UVC exposure. In some embodiment, a parameter for each family of biomolecules may help estimate the effectiveness of disinfectant systems for any virus whose structure is known. In a family of viruses such as single strand RNA viruses (or other families of viruses), the number of bases is approximately constant, i.e., independent of the exact number of bonds that constitute the virus. The effectiveness of UVC systems against “known and unknown viruses” may then be estimated.

In a recent study, two samples of COVID-19 viruses were injected in an aerosol irradiation chamber and exposed to about 222 nm UVC light. The inactivation followed a typical exponential disinfection model, with a fixed inactivation constant for each of the two COVID-19 viruses. The study has shown that UV light doses of approximately ˜1.7 mJ/cm2 (about one (1) second exposure to 1.7 mWatt/cm2 UVC) produces proximately 99.9% inactivation (3-log reduction). In one embodiment, the numbers were used as guideline for the design of the disinfection system.

A basic difference between a UVC based decontamination chamber for compressible matter in contrast to other systems which are used for disinfection of incompressible liquids such as water, is that incompressible fluids need to be stored in UVC for a length of time. On the other hand, flow of compressible matter—such as air—may be controlled by management of pressure profiles in the path of the air. In one embodiment, programmable air flow control systems may be a part of the disinfection area itself or these may precede the disinfection area as a separate component of the system. Steady air flow in the disinfection area may be achieved by creating a laminar flow using filters like, but not limited to, HEPA filters. And/Or, using specially designed tubes with bends and surface finishes which may slow air velocity prior to the entry of air into the disinfection area. Some of these techniques are used to produce controlled air flow such as “clean air benches”. For air disinfection systems, a “low rate of flow” may be enabled so that the transit time through the disinfection area is long. In one embodiment, air flow control function may be integrated with the design of the disinfection area. Air flow control function may be achieved by using filters, or by utilizing geometrical features in the path of the air flow to create a step change in pressure, or by introducing impedances in the path of air flow by dense packing of UVC transparent material to produce a pressure distribution and by using air compressors to produce pressure or a combination of these items.

In additional embodiment, multiple safety mechanism for protecting users from direct UVC radiation may be implemented. Multiple safety mechanisms may include, but not limited to, power supply disabling switches in case of intentional or unintentional dismantling of the decontamination chamber. Sensors to detect leakage of UVC radiation. Means to disable power supply to the UVC source. Eye wear that blocks the UV wavelengths contained in the LED lights. For high pressure systems, pressure relief valves may be used in areas where air pressure may be higher than atmospheric pressure. A remote controller may be incorporated in the disinfection system to allow the disinfection system to be turned on/off at a distance to avoid exposure to UV rays produced in the decontamination chamber.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” or “steps for” claim limitation implies that the broadest initial search on 35 USC § 112(6) (post AIA 112(f)) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112(6) (post AIA 112(f)) when such corresponding structures are not explicitly disclosed in the foregoing patent specification. Therefore, for any invention element(s)/structure(s) corresponding to functional claim limitation(s), in the below claims interpreted under 35 USC § 112(6) (post AIA 112(f)), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claim's construction proceedings and/or examination of patent allowability properly identify and incorporate only the portions of each of these documents discovered during the broadest interpretation search of 35 USC § 112(6) (post AIA 112(f)) limitation, which exist in at least one of the patents and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112(6) (post AIA 112(f)), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing disinfection system according to the present invention will be apparent to those skilled in the art. Various aspects of the invention have been described above by way of illustration, and the specific embodiments disclosed are not intended to limit the invention to the particular forms disclosed. The particular implementation of the disinfection system may vary depending upon the particular context or application. By way of example, and not limitation, the disinfection system described in the foregoing were principally directed to air disinfection implementations; however, similar techniques may instead be applied to air purification of air in confined or sterile spaces, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

Only those claims which employ the words “means for” or “steps for” are to be interpreted under 35 USC 112, sixth paragraph (pre-AIA) or 35 USC 112(f) post-AIA. Otherwise, no limitations from the specification are to be read into any claims, unless those limitations are expressly included in the claims.

What is claimed is:

1. A disinfection system comprising:
an air mover, in which said air mover comprises at least one of a fan and air compressor that is configured to direct air flow;
an inlet flow control valve, in which said inlet flow control valve comprises a programmable flow control valve that is configured to turn off to obstruct air flow and to turn on to allow the flow of air;
a decontamination chamber apparatus, in which said decontamination chamber apparatus comprises an inlet section or port, an inner wall, and an outlet section or port;
in which said decontamination chamber apparatus further comprises about two or more decontamination chambers;
in which each of said two or more decontamination chambers comprises a plurality of decontamination areas;
a plurality of UVC transparent materials, said plurality of UVC transparent materials are configured to be operable for impeding/obstructing the flow of air in each of said plurality of decontamination areas;
a UV light source, wherein said UV light source is configured to produce light in the UVC wavelength, wherein said UV light is configured to disinfect air that goes through said decontamination chamber apparatus;
an outlet flow control valve, wherein said outlet flow control valve is configured to recirculate the disinfected air;
a programmable controller, wherein said programmable controller is configured to be operable for slowing-down/halting the flow of air; and
a timer for determining an amount of time for slowing-down/halting the flow of air.

2. A disinfection system comprising:
an air mover, wherein said air mover is configured to direct air flow;
an inlet filter implement;
an inlet flow control valve;
a decontamination chamber apparatus, in which said decontamination chamber apparatus comprises an inlet section or port, an inner wall, and an outlet section or port;
a UV light source, wherein said UV light source is configured to produce light in the UV wavelength, wherein said UV light is configured to disinfect air that passes through said decontamination chamber apparatus;
an outlet flow control valve, wherein said outlet flow control valve is configured to recirculate the disinfected air; and
wherein said decontamination chamber apparatus is configured to be operable for impeding/obstructing the flow of air.

3. The system of claim 2, further comprising a controller that is configured to turn on/off at least one of said air mover, inlet flow control valve, and outlet flow control valve to slow-down/halt the flow of air.

4. The system of claim 3, in which said air mover comprises at least one of, a fan and an air compressor.

5. The system of claim 4, in which said inner wall comprises a reflective material for diffusing incident UV light striking said reflective material.

6. The system of claim 4, in which said UV light source comprises at least one of, UVC LED, Hg arc lamp, pulsed Xenon lamp, and K-Br lamp.

7. The system of claim 5, in which said reflective material comprises at least one of an aluminum material deposited, plated, or coated on said inner wall, and aluminum foil glued to said inner wall.

8. The system of claim 5, in which said reflective material comprises at least a Polytetrafluoroethylene material deposited, coated, or plated on said inner wall.

9. The system of claim 7, in which said aluminum or aluminum foil material is covered with a dielectric material to protect said reflective material.

10. The system of claim 5, in which said decontamination chamber apparatus further comprises at least one or more decontamination areas.

11. The system of claim 10, in which each of said at least one or more decontamination areas is filled with impeding/obstructing material including at least one of, UVC transparent glass balls, powdered Quartz, and Quartz wool configured to impede, obstruct, or hinder the flow of air.

12. The system of claim 11, in which said at least one or more decontamination areas are separated with filters.

13. The system of claim 12, in which said at least one or more decontamination areas are separated with filters.

14. The system of claim 13, wherein said air compressor builds pressure on said inlet section or port to enable a movement or air through said decontamination areas with obstructions and filters.

15. The system of claim 3, in which said controller comprises at least one of, a programmable controller, a program and processor, and a computer that is configured to automatically turn on/off at least one of said air mover, inlet flow control valve, and outlet flow control valve to slow-down/halt the flow of air at predetermined times and conditions.

16. The system of claim 1, in which said decontamination chamber apparatus comprises multiple decontamination chamber apparatus connected in series to enhance the level of disinfection.

17. The system of claim 2, in which said decontamination chamber apparatus comprises multiple decontamination chamber apparatus connected in parallel to enhance the level of disinfection.

18. A disinfection system comprising:
means for directing air flow;
an inlet filter implement;
means for obstructing air flow;
means for disinfecting air mass;
in which said air mass disinfecting means comprises about two more air mass disinfecting means;
means for impeding/obstructing the flow of air in said air mass disinfecting means;
means for producing light in the UVC wavelength, wherein said UVC light is configured to disinfect air that goes through said air mass disinfecting means;
means for recirculating the disinfected air; and
means for slowing-down/halting the flow of air.
